(19) United States
(12) Patent Application Publication
(10) Pub. No.: US 2025/0235686 A1
(43) Pub. Date: Jul. 24, 2025

(54) RIGHT VENTRICULAR ASSIST DEVICE

(71) Applicant: EDWARDS LIFESCIENCES CORPORATION, Irvine, CA (US)

(72) Inventors: Emil Karapetian, Huntington Beach, CA (US); Arnold Cruz Tuason, Claremont, CA (US); Sam Chitsaz (formerly Chitsaz Avval), Walnut Creek, CA (US); Jenna Christine Iaizzo, San Clemente, CA (US)

(73) Assignee: EDWARDS LIFESCIENCES CORPORATION, Irvine, CA (US)

(21) Appl. No.: 19/173,459

(22) Filed: Apr. 8, 2025

Related U.S. Application Data
(63) Continuation of application No. PCT/US2023/076346, filed on Oct. 9, 2023.
(60) Provisional application No. 63/378,922, filed on Oct. 10, 2022.

(51) Int. Cl. A61M 60/117; A61M 60/531; A61M 60/845

(52) U.S. Cl. CPC A61M 60/117; A61M 60/531; A61M 60/845; A61M 2205/18; A61M 2205/3334

(57) ABSTRACT

A right ventricular assist device (hereinafter, “RVAD”) for placement inside a mammalian subject includes a tube having an inlet configured to be positioned in the mammalian subject when the tube is in a proper position within a heart and an outlet configured to be located in a pulmonary artery when the tube is in the proper position. The RVAD also includes a plurality of sensors having measurement locations along the tube to measure a plurality of pressures of blood exterior to the tube at the measurement locations and a controller in communication with the pump and the plurality of sensors that convey the plurality of pressures to the controller with the controller being configured to instruct the pump to pump blood through the tube depending on at least one of the plurality of pressures.

### CROSS-REFERENCE TO RELATED APPLICATION(S)

This application is a continuation of PCT International Application No. PCT/US2023/076346, filed Oct. 9, 2023, which claims the benefit of U.S. Provisional Application No. 63/378,922, filed Oct. 10, 2022, the disclosures of which are hereby incorporated by reference in their entireties.

### BACKGROUND

The present disclosure relates generally to heart catheters and, more particularly, to a right ventricular assist device (hereinafter, “RVAD”).

Patients with pulmonary hypertension and/or right ventricular dysfunction have higher morbidity and mortality rates when undergoing procedures requiring anesthesia. Cardiac assist is often provided in peri-operative settings to help a right ventricle of the heart avoid excess loading that can cause right heart failure and/or worsen right ventricle dysfunction. Additionally, cardiac assist can be provided to the right ventricle in patients suffering from pulmonary embolism and/or right heart failure.

Current cardiac assist devices used in the right side of the heart have many drawbacks. Inserting and positioning the devices correctly within the right side of the heart is difficult because the device has to serpentine through the right atrium, tricuspid valve, right ventricle, and pulmonic valve to access the pulmonary artery. Thus, the devices are prone to misplacement. The devices are also prone to dislodgement/movement after insertion. Additionally, the presences of a cardiac assist device in the right side of the heart extending through the tricuspid valve and the pulmonary valve can cause those valves to remain at least partially continuously open, resulting in additional stress on the right side of the heart.

### SUMMARY

A right ventricular assist device (hereinafter, “RVAD”) is disclosed herein that uses sensors positioned along a tube of the RVAD to monitor the placement and ensure the RVAD is properly positioned within the right side of the heart. These sensors are used in conjunction with a controller that, depending on the pressures measured by the sensors along the RVAD, can 1) trigger an alert to notify medical personnel that the RVAD is misplaced; 2) increase or decrease a flow of blood pumped by the RVAD to overcome valve dysfunction and/or maintain a pressure of blood within the pulmonary artery or right ventricle above or below a threshold pressure; and 3) activate one or more spacers positioned within/adjacent the tricuspid valve and/or pulmonary valve to plug any gaps formed within the valves due to the presence of the RVAD and/or dysfunction of the valves.

### BRIEF DESCRIPTION OF THE DRAWINGS

FIG. 1 is a perspective view of an RVAD.

FIG. 2A is a cross-sectional view of a mammalian heart with a distal tip of the RVAD in the right atrium.

FIG. 2B is a cross-sectional view of the mammalian heart with the distal tip of RVAD in the right ventricle.

FIG. 2C is a cross-sectional view of the mammalian heart with the distal tip of the RVAD in the pulmonary artery such that the RVAD is properly position.

FIG. 3A is a cross-sectional view of a mammalian heart with an example of the RVAD with spacers.

FIG. 3B is an enlarged cross-sectional view of a heart valve with the RVAD extending therethrough without the spacer deployed.

FIG. 3C is the heart valve of FIG. 3B with the spacer deployed.

FIG. 4 is a cross-sectional view of a mammalian heart with another example of the RVAD with spacers.

### DETAILED DESCRIPTION

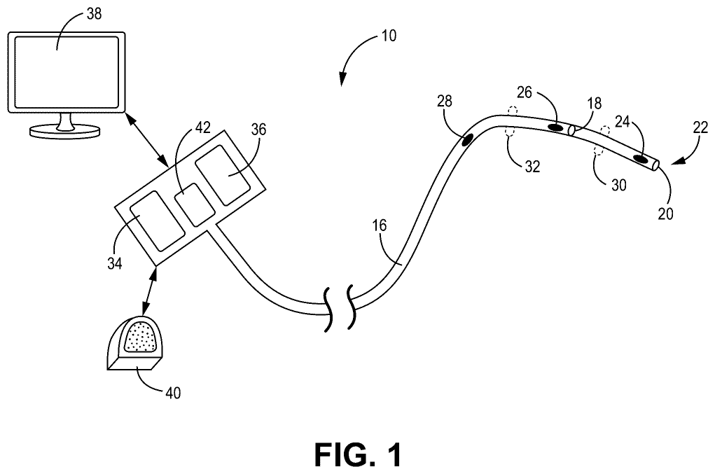

FIG. 1 is a perspective view of RVAD 10 in communication with display device 38 and alert system 40. RVAD 10 includes tube 16, inlet 18, outlet 20 at distal end 22, first sensor 24, second sensor 26, third sensor 28, first spacer 30, second spacer 32, pump 34, controller 36, display device 38, alert system 40, and optional sensor housing 42. While first spacer 30 and second spacer 32 are shown as bulges along tube 16, spacers 30 and 32 can be configured such that each one is contracted when not in use (e.g., when being inserted into the heart) so as to have a similar diameter/cross-sectional shape to tube 16 and extend to have a greater diameter/cross-sectional shape than tube 16 (e.g., to be bulges) when in use to plug gaps in the vales of the heart. So as to show position along tube 16, spacers 30 and 32 are shown as bulges even though they may not be extended during the insertion and operation of RVAD 10.

RVAD 10 is an example of a cardiac assist device configured to be inserted into a heart of a mammalian subject, such as a human, without the need for open heart surgery. RVAD 10 (specifically, tube 16) is typically inserted into the heart for temporary use to case loading on the right ventricle of the heart. RVAD 10 is positioned to pump blood from the right ventricle to the pulmonary artery. Inserting/positioning RVAD 10 within the right side of the heart can be difficult because RVAD 10 needs to serpentine through the right atrium, the tricuspid valve, the right ventricle, and the pulmonary valve to access the pulmonary artery. Thus, catheter-based cardiac assist devices can be prone to misplacement (without the features disclosed herein that reduce misplacement). Cardiac assist devices can also be prone to dislodgement/movement after insertion (without the features disclosed herein that reduce dislodgement/movement). Additionally, the presence of cardiac assist devices extending through the tricuspid valve and the pulmonary valve may cause those valves to remain at least partially continuously open during the open-close cycle of the heart. However, RVAD 10 can include features, such as sensors 24-28, to detect if the valves are open and first spacer 30 and second spacer 32 to plug gaps in the valves when the valves are at least partially continuously open.

RVAD 10 uses sensors (first sensor 24, second sensor 26, and third sensor 28) along tube 16 to monitor the placement and ensure RVAD 10 is properly positioned within the right side of the heart (or, in addition to the being positioned within the heart, a portion of RVAD 10 can be positioned within the vena cava or other components of the human subject). Sensors 24-28 are used in conjunction with controller 36 that, depending on the pressures measured by sensors 24-28 along tube 16, can 1) trigger an alert by alert system 40 (and potentially by display device 38) to notify medical personnel that RVAD 10 is misplaced; 2) increase or decrease a flow of blood pumped by pump 34 of RVAD 10 to overcome valve dysfunction and/or maintain a pressure of blood within the pulmonary artery or right ventricle above or below a threshold pressure; and 3) activate one or more spacers 30 and 32 positioned within/adjacent the tricuspid valve and/or pulmonary valve to plug any gaps formed within the valves due to the presence of RVAD 10 and/or dysfunction of the valves.

Alternatively and/or additionally, sensors 24-28 can be located within sensor housing 42 distant from distal end 22 of tube 16, and tube 16 can include ports each located at measurement locations shown as reference numbers 24, 26, and 28 in FIG. 1. The ports can each be fluidically connected to sensors in sensor housing 42 by individual ducts that extend at least partially within tube 16 to convey the blood pressure at each measurement location at 24, 26, and 28 to each sensor for measurement. Such a configuration enables the sensors to be positioned, not within tube 16, but instead distant from and outside of tube 16.

While RVAD 10 is described herein as being used within the right side of a heart, other configurations can include RVAD 10 that is included in the left side of the heart or other locations. Also, RVAD 10 can include other components, features, functions, and capabilities not expressly disclosed herein, and the components and features disclosed can be in any configuration suitable for accomplishing the functions of RVAD 10. RVAD 10 can be constructed from any material or combination of materials that are suitable for use within and/or near living subjects (e.g., living mammalian tissue: arteries, veins, organs, etc.). RVAD 10 can be constructed so as to be single use (used once and then discharged after being removed from the mammalian subject) or multiple use (used multiple times on one or multiple mammalian subjects). The components of RVAD 10 that may come into contact with the mammalian subject can be sterilized to remove any contaminants/germs on those components, and RVAD 10 can be sterilized before use/insertion into the mammalian subject.

RVAD 10 includes tube 16, which can be a flexible cylinder that is configured to extend from a location outside the mammalian subject to distal end 22 of tube 16 located within the pulmonary artery of/adjacent the heart. Tube 16 can have any cross-sectional shape, but a cross-sectional shape without sharp edges (such as circular, oblong, or oval) may be preferred so as to not damage the mammalian subject when tube 16 is inserted into and removed from the heart. Additionally, tube 16 should have a cross-sectional area (i.e., flow area) to allow for sufficient movement/pumping of blood from inlet 18 to outlet 20. However, the cross-sectional area of tube 16 should not be too large so as to be difficult or prevent the insertion and removal of tube 16 into and out of the heart, respectively. Tube 16 can be partially or mostly hollow, or tube 16 can house other components of RVAD 10. Tube 16 can be as short or long as necessary to allow for insertion into the heart from outside the body of the mammalian subject. Tube 16 can be fluidically, electrically, and/or wirelessly connected to components that can be configured to remain outside the body at all times. These components can be, but do not have to be, sensors 24-28, controller 36, display device 38, alert system 40, and/or sensor housing 42. Thus, tube 16 can, for example, include one or multiple lumens for fluidically or electrically connecting components on tube 16 to components configured to remain outside the body. Tube 16 can also include one or multiple electrical lines/wires extending through one or multiple lumens (that extend from sensors 24-28 in the example where sensors 24-28 are located within tube 16 near distal end 22, extend from a power source to pump 34 in the example where pump 34 is located within tube 16, and/or extend from one or multiple components in tube 16 to controller 36, display device 38, and/or alert system 40). Tube 16 can also include one or multiple fluid lumens (that extend from measurement locations shown as reference numbers 24, 26, and 28 in FIG. 1 to sensor housing 42 in the example where the sensors are positioned in sensor housing 42, that extend from inlet 18 to outlet 20, and/or that extend from inlet 18 and/or outlet 20 to pump 34 located outside the mammalian subject). Finally, tube 16 can include other components present within or extending therethrough.

Tube 16 includes outlet 20, which is an opening in tube 16, located near or at distal end 22. Additionally, tube 16 includes inlet 18, which is an opening in tube 16, located distant from distal end 22. The distance between inlet 18 and outlet 20 along tube 16 should be sized so that, when tube 16 is in a proper positioned within the heart, inlet 18 is located within the right ventricle and outlet 20 is located within the pulmonary artery. Between inlet 18 and outlet 20 within tube 16 is a lumen or channel (not shown) that allows for blood to be pumped by pump 34 from the right ventricle, through tube 16, and discharged at outlet 20 into the pulmonary artery. Thus, tube 16, with inlet 18, outlet 20, and pump 34, allows for the bypass of blood from the right ventricle to the pulmonary artery without the need for the heart to pump the blood through the pulmonary valve. Therefore, the stress on the heart is reduced. In an alternate embodiment, inlet 18 can be positioned on tube 16 to be located in the right atrium when RVAD 10 is positioned in the heart, and RVAD 10 can pump blood from the right atrium to the pulmonary valve. In another alternate embodiment, inlet 18 can be positioned so as to pull blood from the vascular upstream of the heart. As disclosed earlier, pump 34 can be located outside of the body of the mammalian subject, or pump 34 can be located within tube 16 near inlet 18 and/or outlet 20. Outlet 20 can be located at a tip at distal end 22 such that the opening of outlet 20 is at the tip, or outlet 20 can be located merely along tube 16 but not at the tip at distal end 22 of tube 16.

While tube 16 is shown and described herein as having inlet 18 configured to be located within the right ventricle when outlet 20 is located within the pulmonary artery, other configurations of RVAD 10 can have inlet 18 located in the right atrium of the heart or the vena cava outside the heart to allow blood to be pumped from any of those locations to the pulmonary artery.

As shown in the example of FIG. 1, first sensor 24 (at first measurement location 24), second sensor 26 (at second measurement location 26), and third sensor 28 (at third measurement location 28) are located on and/or within tube 16 near distal end 22 (as compared to the proximal end near pump 34, controller 36, and sensor housing 42 and distant from distal end 22) to measure the pressure of the blood adjacent sensors 24-28, respectively. Each of sensors 24-28 can have any configuration capable of measuring a pressure of the blood adjacent sensors 24-28 along tube 16. Sensors 24-28 can be located within tube 16, and each can have one or multiple openings in tube 16 to convey the blood to the sensor for measurement. Alternatively, sensors 24-28 can be located on an outer surface of tube 16 (or the measurement surface/device of sensors 24-28 can be on the outer surface of tube 16) so that blood is not present within tube 16 for measurement by sensors 24-28 but rather the measurement is performed/collected external to tube 16.

In another example, the three sensors are located within sensor housing 42, which is distant from distal end 22 of tube 16. In this example, three ducts run through tube 16 from ports at each of measurement locations 24, 26, and 28 to each of the first sensor, the second sensor, and the third sensor, respectively, to allow for the pressure of blood to reach the sensors and be measured by the sensors. The ports allow blood to enter the ducts, and the ducts convey the pressure of the blood at measurement locations 24-28 to the sensors, thus eliminating the need for the sensors to be located along/within tube 16.

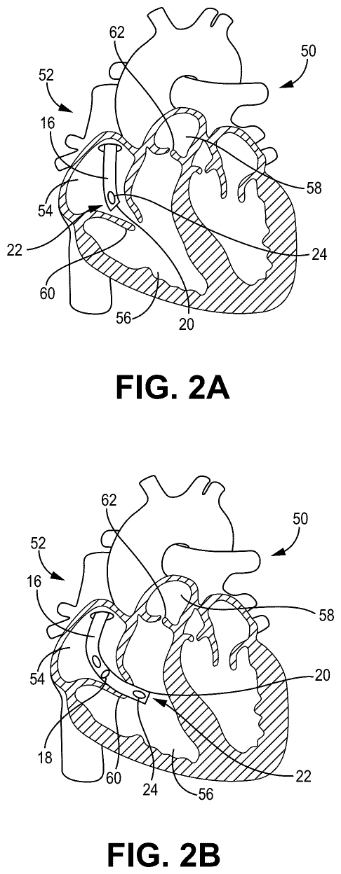

Sensors 24-28 can be in wired or wireless communication with display device 38, which can be configured to display the pressures measured by sensors 24-28. Sensors 24-28 can measure blood pressures once, periodically, or continuously and convey those measurements to display device 38 for visual display for viewing by medical professionals. Additionally, sensors 24-28 can measure and/or record other information/data. Sensors 24-28 can be in communication with controller 36 such that any measurements collected by sensors 24-28 are conveyed to controller 36 for further analysis and/or trigger action by controller 36. Furthermore, controller 36 can control/instruct sensors 24-28 on if/when to measure the blood pressures. Sensors 24-28 can be positioned along tube 16 and spaced apart from one another at the following locations: first sensor 24 is located near distal end 22 of tube 16 (and potentially near outlet 20) such that, when tube 16 is in the proper position within the heart, first sensor 24 is within the pulmonary artery and is able to measure the pressure of blood within the pulmonary artery; second sensor 26 is located distant from first sensor 24 and distal end 22 (and potentially near inlet 18) such that, when tube 16 is in the proper position within the heart, second sensor 26 is within the right ventricle and is able to measure the pressure of blood within the right ventricle; and third sensor 28 is located distant from second sensor 26 such that, when tube 16 is in the proper position within the heart, third sensor 28 is within the right atrium and is able to measure the pressure of blood within the right atrium. The measurement locations of sensors 24-28 are described in more detail below with regards to FIGS. 2A-2C.

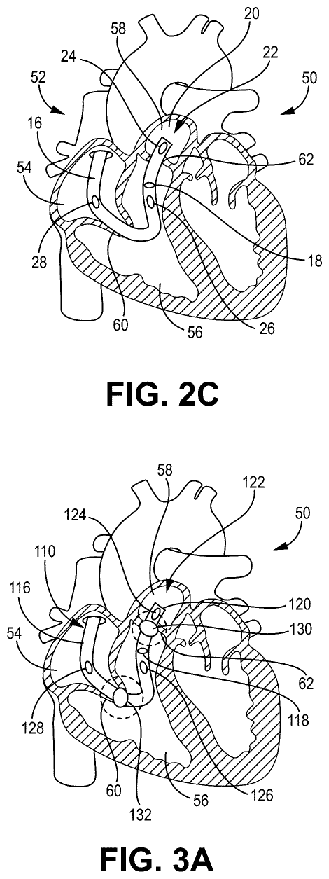

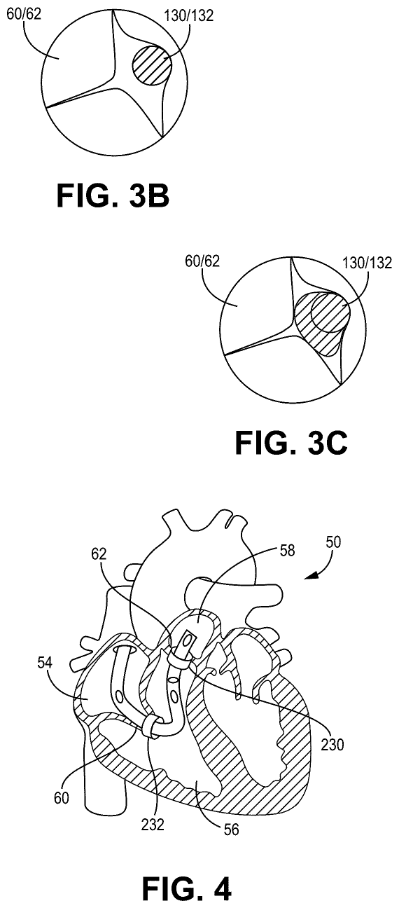

First spacer 30 is located along tube 16 between first sensor 24 and second sensor 26, while second spacer 32 is located along tube 16 between second sensor 26 and third sensor 28. First spacer 30 is positioned such that, when tube 16 is in the proper position within the heart, first spacer 30 is located within or adjacent to the pulmonary valve (also referred to as the first valve) between the pulmonary artery and the right ventricle. First spacer 30 is configured to plug a gap in the pulmonary valve during the instance when the pulmonary valve is at least partially continuously open. Similarly, second spacer 32 is positioned such that, when tube 16 is in the proper position within the heart, second spacer 32 is located within or adjacent to the tricuspid valve (also referred to as the second valve) between the right ventricle and the right atrium. Second spacer 32 is configured to plug a gap in the tricuspid valve during the instance when the tricuspid valve is at least partially continuously open. The two valves may be at least partially continuously open due to tube 16 extending through the valves, preventing the valves from completely closing/sealing. The failure of the valves to completely close can cause additional stresses on the heart. Thus, first spacer 30 and second spacer 32 can be configured to plug a gap in each valve, respectively. Spacers 30 and 32 can have any configuration suitable for plugging a gap in a valve. Some examples of first spacer 30 and second spacer 32 are shown and described with regards to FIGS. 3A-3C and 4, with spacers 30 and 32 able to be a variety of devices, including an inflatable balloon, a covered braid, a covered wireform, and a temporary valve. Additionally, RVAD 10 can be configured to include one spacer, more than two spacers, or no spacers at all.

Pump 34 can be any type of device configured to move blood from inlet 18 to outlet 20. Pump 34 can be a reciprocating, rotary, or another type of pump that can provide a pressure differential such that blood is drawn into inlet 18 from the right ventricle when tube 16 is in the proper position, flows through tube 20, and is pushed out of outlet 20 into the pulmonary artery when tube 16 is in the proper position. Pump 34 can be located within tube 16 between inlet 18 and outlet 20, at another location within tube 16, or outside of tube 16, such as at the proximal end of tube 16 distant from distal end 22. Pump 34 can be capable of varying speeds to vary the flow rate of blood through tube 16, or pump 34 can be capable of a constant speed to maintain a constant flow rate of blood through tube 16. The rate at which pump 34 pumps blood through tube 16 can be determined depending on measurements collected by sensors 24-28 and/or other factors. Pump 34 can be in communication with controller 36 to receive instructions from controller 36 regarding if/when to turn on and off, the rate at which to pump blood through tube 16, and other functions.

Controller 36 can include one or multiple computer/data processors. In general, the computer/data processors can include any or more than one of a processor, a microprocessor, a controller, a digital signal processor (DSP), an application specific integrated circuit (ASIC), a field-programmable gate array (FPGA), or other equivalent discrete or integrated logic circuitry. Controller 36, and RVAD 10 as a whole, can include other components not expressly disclosed herein but that are suitable for performing the functions of RVAD 10 and associated methods. For example, controller 36 (and RVAD 10) can include communication software and/or hardware for communicating with other components of RVAD 10 and with components/systems distinct from RVAD 10. Controller 36 can be a physical component contained within RVAD 10, or controller 36 can be distant from RVAD 10, such as a cloud-based component/system or a system in conjunction (e.g., incorporated with) display device 38 and/or alert system 40. Additionally, controller 36 (and RVAD 10) can include machine-readable storage media. In some examples, a machine-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, storage media can be entirely or in part a temporary memory, meaning that a primary purpose storage media is not long-term storage. Storage media, in some examples, is described as volatile memory, meaning that the memory, does not maintain stored contents when power to RVAD 10 and/or controller 36 (or the component(s) where storage media are located) is turned off. Examples of volatile memories can include random access memories (RAM), dynamic random-access memories (DRAM), static random-access memories (SRAM), and other forms of volatile memories. In some examples, storage media can also include one or more machine-readable storage media. Storage media can be configured to store larger amounts of information than volatile memory. Storage media can further be configured for long-term storage of information. In some examples, storage media include non-volatile storage elements. Examples of such non-volatile storage elements can include magnetic hard discs, optical discs, flash memories and other forms of solid-state memory, or forms of electrically programmable memories (EPROM) or electrically erasable and programmable (EEPROM) memories. Most generally, storage media is machine-readable data storage capable of housing stored data from a stored data archive.

Controller 36 can be in communication with sensors 24-28 to receive measurement data collected by each of sensors 24-28 and in communication with pump 34 to instruct pump 34 to pump blood from inlet 18 to outlet 20. Controller 36 can instruct pump 34 to pump at a constant rate (i.e., the blood flows through tube 16 at a constant rate), at a variable rate (i.e., the blood flows through tube 16 at a varying rate), and/or on a schedule that includes both constant and variable rates.

Controller 36 can instruct pump 34 to pump blood depending on the measurements (e.g., the measurements of blood pressures at each of the measurement locations) collected by sensors 24-28 and other factors. For example, controller 36 can be configured to instruct pump 34 to vary the flow rate of blood to maintain the pressure of blood in the pulmonary artery (as measured by first sensor 24) above a first threshold pressure, which can be set by the medical professional dependent upon the physiology of the mammalian subject. Controller 36 can also be configured to instruct pump 34 to vary the flow rate of blood to maintain the pressure of blood in the pulmonary artery (as measured by first sensor 24) below a second threshold pressure, which can be set by the medical professional dependent upon the physiology of the mammalian subject. The first and second threshold pressures can be dependent upon a baseline and can trigger an alert if the threshold pressures deviate from the baseline by a preset amount (e.g., a percentage). Additionally, controller 36 can be configured to trigger an alert if the pressure waveform/profile changes to a waveform/profile that is indicative of suboptimal positioning of RVAD 10. Controller 36 can be configured to continuously monitor the pressure measurements collected by sensors 24-28 for any pressure inconsistencies and discrepancies. For example, controller 36 can continuously monitor the pressure measurement collected by sensors 24-28 for changes that indicate that first sensor 24 is not positioned in the pulmonary artery, second sensor 26 is not in the right ventricle, and/or third sensor 28 is not in the right atrium. Controller 36 can be configured to compare the pressure measurements collected by sensors 24-28 to one another for an indication that any of sensors 24-28 are not properly positioned. For example, controller 36 can compare the blood pressure measured by first sensor 24 to the blood pressure measured by second sensor 26 and, in response to the blood pressure measured by first sensor 24 being within a first margin relative to the blood pressure measured by second sensor 26, determine that first sensor 24 is improperly positioned in the same heart chamber (i.e., the right ventricle) as second sensor 26 and/or that the pulmonary valve is at least partially continuously open. Similarly, controller 36 can compare the blood pressure measured by second sensor 26 to the blood pressure measured by third sensor 28 and, in response to the blood pressure measured by second sensor 26 being within a second margin relative to the blood pressure measured by third sensor 28, determine that second sensor 26 is improperly positioned in the same heart chamber (i.e., the right atrium) as third sensor 28 and/or that the tricuspid valve is at least partially continuously open.

Controller 36 can also be configured to determine a first pressure waveform from the measurement of blood pressure by first sensor 24. The first pressure waveform can be compared to a normal, baseline pressure waveform of the pressure within the pulmonary artery of a properly functioning heart. If the first pressure waveform is different from the normal pressure waveform of the pulmonary artery of a properly functioning heart, controller 36 can determine that the pulmonary valve is at least partially continuously open and/or first sensor 24 is improperly positioned and is not within the pulmonary artery (and thus outlet 20 is also not within the pulmonary artery). Controller 36 can be configured to determine other results from first pressure waveform, such as other issues with the heart. Similarly, controller 36 can be configured to determine a second pressure waveform from the measurement of blood pressure by second sensor 26. The second pressure waveform can be compared to a normal, baseline pressure waveform of the pressure within the right ventricle of a properly functioning heart. If the second pressure waveform is different from the normal pressure waveform of the right ventricle of a properly functioning heart, controller 36 can determine that the pulmonary valve and/or tricuspid valve is/are at least partially continuously open and/or second sensor 26 is improperly positioned and is not within the right ventricle (and thus inlet 18 is also not within the right ventricle). Similarly, controller 36 can be configured to determine a third pressure waveform that can be compared to a normal, baseline pressure waveform of the pressure within the right atrium of a properly functioning heart. If the third pressure waveform is different from the normal pressure waveform of the right atrium of a properly functioning heart, controller 36 can determine that the tricuspid valve is at least partially continuously open and/or third sensor 28 is improperly positioned and is not within the right atrium. Controller 36 can be configured to determine other results from the first, second, and third pressure waveforms, such as other issues with the heart.

Controller 36 can be in communication with each of first spacer 30 and second spacer 32. Controller 36 can be configured to instruct each of spacers 30 and 32 to plug a gap in the pulmonary valve or tricuspid valve, respectively. Controller 36 can instruct spacers 30 and 32 to plug the gaps depending on the measurements (e.g., the measurements of blood pressures at each measurement location) collected by sensors 24-28 and other factors. Controller 36 can be configured to, from the measurements of blood pressures collected by sensors 24-28, determine a variety of data, and controller 36 can depend on that data for instructing spacers 30 and 32 to plug the gaps in the valves. Controller 36 can determine a first pressure gradient from the measurement of blood pressures by first sensor 24 and second sensor 26, and determine a second pressure gradient from the measurement of blood pressures by second sensor 26 and third sensor 28. Controller 36 can determine if the pulmonary valve and/or the tricuspid valve are at least partially continuously open from these pressure gradients. The first pressure gradient can be indicative of a situation where the pulmonary valve is at least partially continuously open, so controller 36 can instruct first spacer 30 to plug the gap in the pulmonary valve in response to such indication/determination from the first pressure gradient. Similarly, the second pressure gradient can be indicative of a situation where the tricuspid valve is at least partially continuously open, so controller 36 can instruct second spacer 32 to plug the gap in the tricuspid valve in response to such indication/determination from the second pressure gradient. The pressure gradients can continuously be determined and monitored for any inconsistencies or discrepancies that would indicate that either of the valves are at least partially continuously open.

Controller 36 can be in communication with display device 38 to display any information received from sensors 24-28, any information received from spacers 30 and 32, and/or any information determined/calculated by controller 36, such as pressures, pressure gradients, pressure waveforms, pressure levels vs. time, and any other information that may be of use to a medical professional. Display device 38 can be in communication with other components of RVAD 10, such as sensors 24-28, spacers 30 and 32, pump 34, and/or alert system 40. Display device 38 can be configured to visually display at least one of the measurements of blood pressure by sensors 24-28 (and/or other information), and can be any device suitable for displaying information for visual viewing by a medical professional. Additionally, display device 38 can be interactive and include touch-sensitive or other buttons for changing the information displayed. Display device 38 can be in wired or wireless communication with controller 36 and/or the other components of RVAD 10, and display device 38 and controller 36 can be incorporated into one system such that the two components share a housing, hardware, and/or software. Additionally, display device 38 can include (i.e., be incorporated into one system sharing a housing, hardware, and/or software) with alert system 40. Display device 38 can be any type of screen able to display information received from controller 36 (and potentially other sources). Display device 38 can include a visual alert system configured to get the attention of medical professionals in response to controller 36 determining that any of first sensor 24, second sensor 26, and third sensor 28 are improperly positioned and/or the pulmonary valve and the tricuspid valve are at least partially continuously open.

Alert system 40 is in communication with controller 36, and can be in communication with other components of RVAD 10, such sensors 24-28, spacers 30 and 32, pump 34, and/or display system 38. Alert system 40 is configured to trigger a visual, audio, and/or other alert in response to controller 36 determining that any of first sensor 24, second sensor 26, and third sensor 28 are improperly positioned and/or the pulmonary valve and the tricuspid valve are at least partially continuously open. Alert system 40 can be incorporated into display system 38 and/or can include its own display, audio speakers, and other features capable of alerting medical professionals. For example, alert system 40 can include wired or wireless communication to send a mobile phone message to a medical professional's mobile phone and/or pager. The alerts triggered by alert system 40 can be the same visual, audio, and/or other alert across all situations (e.g., the same audio sound is triggered when first sensor 24 is improperly positioned and when the pulmonary valve is at least partially continuously open), or the alerts triggered can be different visual, audio, and/or other alert depending on the situation (e.g., one sound is triggered when first sensor 24 is improperly positioned and another sound is triggered when the pulmonary valve is at least partially continuously open). Alert system 40 can trigger alerts depending on or in response to any data collected or determined by any component of RVAD 10.

For example, alert system 40 can be configured to trigger an alert in response to a comparison of the blood pressure measured by first sensor 24 to the blood pressure measured by second sensor 26 revealing that the blood pressure measured by first sensor 24 is within a first margin as compared to the blood pressure measured by second sensor 26 (e.g., this would indicate that first sensor 24 and second sensor 26 are improperly positioned within the same chamber of the heart). The alert can signal that tube 16 is not in the proper position within the heart and/or that the pulmonary valve is at least partially open.

In another example, alert system 40 can be configured to trigger an alert in response to a comparison of the blood pressure measured by second sensor 26 to the blood pressure measured by third sensor 28 revealing that the blood pressure measured by second sensor 26 is within a second margin as compared to the blood pressure measured by third sensor 28 (e.g., this would indicate that second sensor 26 and third sensor 28 are improperly positioned within the same chamber of the heart). The alert can signal that tube 16 is not in the proper position within the heart and/or that the tricuspid valve is at least partially continuously open.

Alert system 40 can be configured to trigger an alert in response to a comparison of a first pressure waveform based on the blood pressure measured by first sensor 24 to a second pressure waveform based on the blood pressure measured by second sensor 26 revealing that the first pressure waveform is within a first waveform margin as compared to the second pressure waveform. Similarly, alert system 40 can be configured to trigger an alert in response to a comparison of the first pressure waveform based on the blood pressure measured by first sensor 24 to a normal, baseline pressure waveform of the pressure within the pulmonary artery of a properly functioning heart revealing that the first pressure waveform is different from the normal pressure waveform of the properly functioning pulmonary artery. The alert can signal that tube 16 is not in the proper position within the heart due to first sensor 24 and second sensor 26 being within the same heart chamber (i.e., the right ventricle), the first sensor 24 having a measured pressure that is inconsistent with a properly functioning pulmonary artery/heart, and/or that the pulmonary valve is at least partially continuously open.

Alert system 40 can be configured to trigger an alert in response to a comparison of the second pressure waveform based on the blood pressure measured by second sensor 26 to a third pressure waveform based on the blood pressure measured by third sensor 28 revealing that the second pressure waveform is within a second waveform margin as compared to the third pressure waveform. Similarly, alert system 40 can be configured to trigger an alert in response to a comparison of the second pressure waveform based on the blood pressure measured by second sensor 26 to a normal, baseline pressure waveform of the pressure within the right ventricle of a properly functioning heart revealing that the second pressure waveform is different from the normal pressure waveform of the properly functioning right ventricle. The alert can signal that tube 16 is not in the proper position within the heart due to second sensor 26 and third sensor 28 being within the same heart chamber (i.e., the right atrium), the second sensor 26 having a measured pressure that is inconsistent with a properly functioning right ventricle/heart, and/or that the tricuspid valve is at least partially continuously open.

In another example, alert system 40 can be configured to trigger an alert in response to a determination of a first pressure gradient from the blood pressure measured by first sensor 24 and the blood pressure measured by second sensor 26 signaling that the pulmonary valve is at least partially continuously open. Similarly, alert system 40 can be configured to trigger alert in response to a determination of a second pressure gradient from the blood pressure measured by second sensor 26 and the blood pressure measured by third sensor 28 signaling that the tricuspid valve is at least partially continuously open. Alert system 40 can be configured to trigger an alert based on other information from RVAD 10 and/or based on information from systems other than RVAD 10.

RVAD 10, with sensor 24-28, is useful in aiding medical professionals in properly positioning tube 16 (with inlet 18, outlet 20, first spacer 30, and second spacer 32) within the heart. The process of inserting RVAD 10 into the heart is shown in FIGS. 2A-2C, which are cross-sectional views of a mammalian heart at various points during the process of inserting RVAD 10 into the proper position.

FIGS. 2A, 2B, and 2C show heart 50 having right side 52 with right atrium 54 and right ventricle 56, and pulmonary artery 58. Between right atrium 54 and right ventricle 56 is tricuspid valve 60, and between right ventricle 56 and pulmonary artery 58 is pulmonary valve 62. While pulmonary artery 58 may be described herein as being a component of heart 50, it is noted that pulmonary artery 58 is not technically classified as a component of heart 50. However, pulmonary artery 58 is part of the mammalian subject and is external to and not a component of RVAD 10 (as is heart 50).

Beginning with FIG. 2A, distal end 22 of tube 16 with first sensor 24 is inserted into heart 50 and specifically first into right atrium 54. While tube 16 is being inserted into heart 50, pump 34 is not in operation (i.e., is not pumping blood). However, sensors 24-28 can be in operation measuring blood pressure as tube 16 is being inserted into heart 50. The blood pressure measurement collection by sensors 24-28 during the insertion of tube 16 into heart 50 can be continuous or periodic (e.g., after each time tube 16 has moved). For example, when tube 16 is in the position shown in FIG. 2A, first sensor 24 should be measuring a blood pressure within right atrium 54. Because right atrium 54 of heart 50 has a particular blood pressure, gradient, waveform, etc. (e.g., normal, baseline right atrium characteristics), a medical professional can compare the measurements collected by first sensor 24 (and potentially determined by controller 36) to the blood pressure, gradient, waveform, etc. of a normal, baseline (e.g., properly functioning) right atrium to determine if distal end 22 of tube 16 is within right atrium 54. If the data collected by first sensor 24 is similar to that of normal, baseline right atrium characteristics, the medical professional installing RVAD 10 can determine that first sensor 24 is within right atrium 54 and the medical professional needs to insert tube 16 further into heart 50.

Continuing the process of inserting tube 16 into heart 50, FIG. 2B shows distal end 22 of tube 16 with first sensor 24 located in right ventricle 56 of heart 50 and second sensor 26 proximal of first sensor 24. At the situation shown in FIG. 2B, distal end 22 and first sensor 24 have moved from right atrium 54, through tricuspid valve 60, and into right ventricle 56. Additionally, the portion of tube 16 including second sensor 26 has moved into right atrium 54. When tube 16 is positioned as shown in FIG. 2B and first sensor 24 and second sensor 26 are in operation collecting measurements of blood pressure, first sensor 24 should be measuring a blood pressure within right ventricle 56 and second sensor should be measuring a blood pressure within right atrium 54. With right ventricle 56 of heart 50 having a particular blood pressure, gradient, waveform, etc. (e.g., normal, baseline right ventricle characteristics), a medical professional can compare the measurements collected by first sensor 24 (and potentially determined by controller 36) to normal, baseline right ventricle characteristics to determine if distal end 22 of tube 16 is within right ventricle 56. The same is true with second sensor 26 and normal, baseline right atrium characteristics: if the two are similar, the medical professional knows that second sensor 26 is located within right atrium 54. If it is determined that first sensor 24 is within right ventricle 56 and second sensor 26 is within right atrium 54, the medical professional knows that he/she needs to insert tube 16 further into heart 50.

Thus, the process of inserting tube 16 into heart 50 continues, and FIG. 2C shows distal end 22 of tube 16 with first sensor 24 located in pulmonary artery 58 and third sensor 28 proximal to second sensor 26. The placement shown in FIG. 2C is the proper placement of tube 16 of RVAD 10 within heart 50. At the situation shown in FIG. 2C, distal end 22 and first sensor 24 have moved from right ventricle 56, through pulmonary valve 62, and into pulmonary artery 58. Also, second sensor 26 has moved from right atrium 54, through tricuspid valve 60, and into right ventricle 56. Third sensor 28 has moved into right atrium 54. When tube 16 is positioned as shown in FIG. 2C in the proper position within heart 50 and pulmonary artery 58 (and sensors 24-28 are in operation collecting measurements of blood pressure), first sensor 24 should be measuring a blood pressure within pulmonary artery 58, second sensor 26 should be measuring a blood pressure within right ventricle 56, and third sensor 28 should be measuring a blood pressure within right atrium 54. Pulmonary artery 58 has a particular blood pressure, gradient, waveform, etc. (e.g., normal, baseline pulmonary artery characteristics), so the measurements collected by first sensor 24 (and potentially determined by controller 36) can be compared to normal, baseline pulmonary artery characteristics to determine if distal end 22 of tube 16 and first sensor 24 are located within pulmonary artery 58. Thus, a medical professional can use the information/measurements collected by first sensor 24 to determine if tube 16 is in the proper position within heart 50. Similarly, the information/measurements collected by second sensor 26 can be compared to normal, baseline right ventricle characteristics to determine if second sensor 26 is located within right ventricle 56, and the information/measurements collected by third sensor 28 can be compared to normal, baseline right atrium characteristics to determine if third sensor 28 is located within right atrium 58. Thus, it can be determined whether tube 16 is in the proper position within heart 50 and pulmonary artery 58 by the blood pressures measured/collected by sensors 24-28.

The comparison of the measurements collected by sensors 24-28 can be performed by controller 36, which can be configured to store blood pressure, gradients, waveforms, etc. for normal, baseline right atrium, right ventricle, and pulmonary artery characteristics; compare the information/measurements collected by sensors 24-28 to the normal, baseline characteristics; and determine within which chamber of heart 50 each of sensors 24-28 are located. This determination can be displayed and/or audibly announced on displace device 38 and/or alert system 40.

Once in the proper position within heart 50, RVAD 10 can activate pump 34 to move/pump blood through tube 16 from inlet 18 positioned in right ventricle 56 to outlet 20 positioned in pulmonary artery 58. Additionally, the measurements collected by sensors 24-28 can be continuously monitored by controller 36 for changes that indicate that any of sensors 24-28 are not properly positioned (i.e., first sensor 24 is not within pulmonary artery 58, second sensor 26 is not within right ventricle 56, and/or third sensor 28 is not within right atrium 54). If any of sensors 24-28 are determined to be improperly positioned within heart 50 and/or pulmonary artery 58, controller 36 can trigger an alert by alert system 40 to notify medical professionals that adjustment of tube 16 may need to be taken.

The capabilities of RVAD 10 having sensors 24-28 and controller 36 configured to measure the blood pressures and determine wavelengths, pressure gradients, etc. during the insertion of tube 16 into heart 50 provides guidance to medical professionals regarding where within heart 50 and/or pulmonary artery 58 tube 16, and more particularly, distal end 22 is located within heart 50 and/or pulmonary artery 58. These capabilities reduce the likelihood that tube 16 is improper positioned within heart 50 and/or pulmonary artery 58. Additionally, the continuous (or periodic) monitoring of the blood pressures measured by sensors 24-28 and the evaluation of those blood pressures, waveforms, pressure gradients, etc. provide insight and indications into whether tube 16 has dislodged/moved from that proper placement after insertion into heart 50 and pulmonary artery 58. Thus, medical professionals have increased knowledge and notice of the placement of tube 16 of RVAD 10.

FIG. 3A is a cross-sectional view of heart 50 with a second example of RVAD 110 in the proper position. FIG. 3B is an enlarged cross-sectional view of tricuspid valve 60 or pulmonary valve 62 with RVAD 110 extending therethrough without first spacer 130 or second spacer 132 deployed. FIG. 3C is an enlarged cross-sectional view of tricuspid valve 60 or pulmonary valve 62 of FIG. 3B with first spacer 130 or second spacer 132 deployed.

FIGS. 3A-3C show RVAD 110 in the proper position within heart 50 and pulmonary artery 58. RVAD 110 within heart 50 and pulmonary artery 58 includes tube 116 extending into the right side of heart 50 and having inlet 118 and second sensor 126 within right ventricle 56, outlet 120 and first sensor 124 in pulmonary artery 58, third sensor 128 in right atrium 54, first spacer 130 within or adjacent to pulmonary valve 62, and second spacer 132 within or adjacent tricuspid valve 60. RVAD 110 includes other components not shown in FIGS. 3A-3C, including those described with regards to FIGS. 1-2C.

As shown in FIG. 3A, RVAD 110 is properly positioned such that first spacer 130 is located within pulmonary valve 62 and second spacer 132 is located within tricuspid valve 60. First spacer 130 and second spacer 132 are configured to plug a gap in pulmonary valve 62 and tricuspid valve 60, respectively, during the situation when/if valves 60 and/or 62 are at least partially continuously open during the open-close cycle. In a properly functioning heart 50, valves 60 and 62 are timely opened to allow blood to move, in the case of tricuspid valve 60, from right atrium 54 to right ventricle 56 and, in the case of pulmonary valve 62, from right ventricle 56 to pulmonary artery 58. A state in which valves 60 and/or 62 are partially continuously open can occur when leaflets of either of valves 60 and 62 do not close entirely, allowing blood to flow through valves 60 and/or 62 continuously even when valves 60 and/or 62 should be in the “closed” state that prevents blood from flowing through valves 60 and/or 62. Such a situation can cause blood to flow in a reverse direction and/or flow at undesirable periods. Valves 60 and 62 can be caused to remain at least partially continuously open during the open-close cycle by the presence of tube 116 extending through valves 60 and 62, by the dysfunction of valves 60 and/or 62, and/or by other factors. Valves 60 and 62 remaining at least partially continuously open can cause stress on heart 50, resulting in increased risk of complications and/or failure of heart 50. Thus, spacers 130 and 132 reduce stresses and risks by plugging a gap formed in valve 60 and 62, respectively, when necessary.

Spacers 130 and 132 can be positioned anywhere along tube 116 so as to be capable of plugging the gaps in valves 60 and 62, respectively. Additionally, spacers 130 and 132 can have any type of configuration capable of plugging the gaps in valves 60 and 62, respectively. In one example shown in FIG. 4, spacers 230 and 232 are temporary valves having a hollow, cylindrical shape that is within or adjacent to pulmonary valve 62 and tricuspid valve 60, respectively. Spacers 230 and 232 can be any type of temporary (or permanent) valve, and can be made from any type of material that is biocompatible. For example, the temporary valve can have multiple leaflets that open and close to allow blood to flow therethrough and plug the opening between the chambers of heart 50. Another example of spacers 130 and 132 is shown in FIGS. 3B and 3C, which can include an inflatable balloon that is capable of expanding to increase a diameter of tube 116 to plug the gaps in valves 60 and 62, respectively. Spacers 130 and 132 as inflatable balloons allows for the cross-sectional shape of the inflatable balloons to be variable to match the cross-sectional shape of partially open valves 60 and 62, thus providing a seal in valves 60 and 62 to prevent blood from flowing therethrough when the inflatable balloons are inflated/expanded. Additional examples include spacers 130 and 132 being covered braids, covered wireforms, or other configurations known in the art capable of expanding to plug the gaps in valves 60 and 62 and contracting to allow blood to flow through valves 60 and 62.

As described above, spacers 30, 32, 130, 132, 230, and/or 232 can be in communication with controller 36 and/or other components of RVAD 10/110. Controller 36 can be configured to instruct spacers 30, 32, 130, 132, 230, and/or 232 to expand/deploy to plug the gap in valves 60 and/or 62 depending on a variety of factors, including the measurements of blood pressures by sensors 24, 26, 28, 125, 126, and/or 128. Controller 36 can be configured to determine if valves 60 and/or 62 are at least partially continuously open and instruct spacers 30, 32, 130, 132, 230, and/or 232 to deploy to plug the gaps in valves 60 and/or 62.

The disclosed RVAD 10/110 uses sensors 24, 26, 28, 124, 126, and/or 128 along RVAD 10/110 to monitor the placement and ensure RVAD 10/110 is properly positioned within heart 50. Sensors 24-28 and 124-128 are used in conjunction with controller 36 that, depending on the pressures measured by sensors 24-28 and 124-128 along RVAD 10/110, can 1) trigger an alert to notify medical personnel that RVAD 10/110 is misplaced; 2) increase or decrease a flow of blood pumped by pump 34 to overcome valve dysfunction and/or maintain a pressure of blood within pulmonary artery 58 and/or right ventricle 56 above or below a threshold pressure; and 3) activate one or more spacers 30, 32, 130, 132, 230, and 232 positioned within/adjacent tricuspid valve 60 and/or pulmonary valve 62 to plug any gaps formed within valves 60 and/or 62 due to the presence of tube 16/116 of RVAD 10/110 and/or dysfunction of valves 60 and/or 62.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

A right ventricular assist device for placement inside a heart and a pulmonary artery of a mammalian subject is disclosed herein that includes a tube having an inlet configured to be positioned in a right side of the heart when the tube is in a proper position within the heart and the pulmonary artery and an outlet configured to be located in the pulmonary artery when the tube is in the proper position. The RVAD also includes a pump connected to the inlet and configured to pump blood from the inlet to the outlet and a plurality of sensors having measurement locations along the tube to measure a plurality of pressures of blood exterior to the tube at the measurement locations, wherein the measurement locations of the plurality of sensors are spaced apart from one another along the tube such that, when the tube is in the proper position within the heart and the pulmonary artery, at least one measurement location is within each of a right atrium, a right ventricle, and the pulmonary artery. The RVAD further includes a controller in communication with the pump and the plurality of sensors, wherein the plurality of sensors convey the plurality of pressures to the controller, and wherein the controller is configured to instruct the pump to pump blood through the tube depending on at least one of the plurality of pressures.

The RVAD of the preceding paragraph can optionally include, additionally and/or alternatively, any one or more of the following features, configurations and/or additional components:

A further embodiment of the foregoing RVAD, wherein the controller is configured to instruct the pump to pump blood at a constant flow rate.

A further embodiment of the foregoing RVAD, wherein the controller is configured to instruct the pump to vary a flow rate of blood pumped through the tube.

A further embodiment of the foregoing RVAD, wherein the controller is configured to instruct the pump to vary the flow rate of blood to maintain a first pressure representing a pressure of blood in the pulmonary artery above a first threshold pressure.

A further embodiment of the foregoing RVAD, wherein the inlet is configured to be positioned within a right ventricle of the heart.

A further embodiment of the foregoing RVAD, wherein the controller is configured to instruct the pump to vary the flow rate of blood to maintain a first pressure representing a pressure of blood in the pulmonary artery at a constant pressure.

A further embodiment of the foregoing RVAD, wherein the controller is configured to instruct the pump to vary the flow rate of blood to maintain a first pressure representing a pressure of blood in the pulmonary artery below a second threshold pressure.

A further embodiment of the foregoing RVAD, wherein the inlet is configured to be positioned within the right atrium of the heart.

A further embodiment of the foregoing RVAD, wherein the plurality of pressure sensors includes a first sensor positioned along the tube adjacent the outlet, the first sensor configured to be located within the pulmonary artery when the tube is in the proper position within the heart and the pulmonary artery and measure a first pressure of the plurality of pressures.

A further embodiment of the foregoing RVAD, wherein the plurality of pressure sensors includes a second sensor positioned along the tube distant from the first sensor, the second sensor configured to be located within the right ventricle when the tube is in the proper position within the heart and the pulmonary artery and measure a second pressure of the plurality of pressures.

A further embodiment of the foregoing RVAD, wherein the plurality of pressure sensors includes a third sensor positioned along the tube distant from the first sensor and the second sensor, the third sensor configured to be located within the right atrium when the tube is in the proper position within the heart and the pulmonary artery and measure a third pressure of the plurality of pressures.

A further embodiment of the foregoing RVAD, wherein the plurality of sensors includes a first port at a first measurement location within the pulmonary artery when the tube is in the proper position within the heart and the pulmonary artery and a first sensor in fluid communication with the first port and configured to measure a first pressure of blood exterior to the tube at the first port.

A further embodiment of the foregoing RVAD, wherein the first sensor is distant from the first port and the plurality of sensors further includes a first lumen extending at least partially within the tube from the first port to the first sensor.

A further embodiment of the foregoing RVAD, wherein the plurality of sensors further includes a second port at a second measurement location within the right ventricle when the tube is in the proper position within the heart, a second sensor in fluid communication with the second port and configured to measure a second pressure of blood exterior to the tube at the second port, and a second lumen extending at least partially within the tube from the second port to the second sensor.

A further embodiment of the foregoing RVAD, wherein the plurality of sensors further includes a third port at a third measurement location within the right atrium when the tube is in the proper position within the heart, a third sensor in fluid communication with the third port and configured to measure a third pressure of blood exterior to the tube at the third port, and a third lumen extending at least partially within the tube from the third port to the third sensor.

A further embodiment of the foregoing RVAD, wherein the first sensor, the second sensor, and the third sensor are incorporated into one pressure measurement device in communication with the controller, the one pressure measurement device configured to measure at least one of the first pressure, the second pressure, and the third pressure.

A further embodiment of the foregoing RVAD that includes a display device in communication with the controller and configured to visually display a measurement of the plurality of pressures measured by each of the plurality of sensors.

A further embodiment of the foregoing RVAD, wherein the plurality of sensors having measurement locations further includes a first sensor having a first measurement location located within the pulmonary artery when the tube is in the proper position within the heart and the pulmonary artery, the first sensor configured to continuously measure a first pressure of blood; a second sensor having a second measurement location located within the right ventricle when the tube is in the proper position within the heart and the pulmonary artery, the second sensor configured to continuously measure a second pressure of blood; and a third sensor having a third measurement location located within the right atrium when the tube is in the proper position within the heart and the pulmonary artery, the third sensor configured to continuously measure a third pressure of blood.

A further embodiment of the foregoing RVAD, wherein the first pressure, the second pressure, and the third pressure are conveyed from the first sensor, the second sensor, and the third sensor, respectively, to the controller.

A further embodiment of the foregoing RVAD that includes first alert that is triggered by the controller in response to a comparison of the first pressure to the second pressure revealing that the first pressure is within a first margin as compared to the second pressure.

A further embodiment of the foregoing RVAD, wherein the first alert is signaling one of the following: the tube is not in the proper position within the heart and the pulmonary artery, and a first valve between the pulmonary artery and the right ventricle is at least partially continuously open.

A further embodiment of the foregoing RVAD that includes a second alert that is triggered by the controller in response to a comparison of the second pressure to the third pressure revealing that the second pressure is within a second margin as compared to the third pressure.

A further embodiment of the foregoing RVAD, wherein the second alert is signaling one of the following: the tube is not in the proper position within the heart and the pulmonary artery, and a second valve between the right ventricle and the right atrium is at least partially continuously open.

A further embodiment of the foregoing RVAD, wherein the controller is configured to determine at least one of a first pressure waveform from the first pressure, a second pressure waveform from the second pressure, and a third pressure waveform from the third pressure.

A further embodiment of the foregoing RVAD that includes a third alert that is triggered by the controller in response to a comparison of the first pressure waveform to the second pressure waveform revealing that the first pressure waveform is within a first waveform margin as compared to the second pressure waveform.

A further embodiment of the foregoing RVAD that includes a fourth alert that is triggered by the controller in response to a comparison of the second pressure waveform to the third pressure waveform revealing that the second pressure waveform is within a second waveform margin as compared to the third pressure waveform.

A further embodiment of the foregoing RVAD, wherein the controller, depending on the first pressure waveform and the second pressure waveform, determines if a first valve is open between the pulmonary artery and the right ventricle.

A further embodiment of the foregoing RVAD, wherein the controller, depending on the second pressure waveform and the third pressure waveform, determines if a second valve is open between the right ventricle and the right atrium.

A further embodiment of the foregoing RVAD that includes a first spacer positioned along the tube and configured to be between the pulmonary artery and the right ventricle when the tube is in the proper position, the first spacer configured to plug a gap in the pulmonary valve.

A further embodiment of the foregoing RVAD, wherein the first spacer is an inflatable balloon, a covered braid, a covered wireform, or a temporary valve.

A further embodiment of the foregoing RVAD that includes a second spacer positioned along the tube and configured to be between the right ventricle and the right atrium when the tube is in the proper position, the second spacer configured to plug a gap in the tricuspid valve.

A further embodiment of the foregoing RVAD, wherein the second spacer is an inflatable balloon, a covered braid, a covered wireform, or a temporary valve.

A further embodiment of the foregoing RVAD, 31, wherein the controller is in communication with the second spacer and is configured to instruct the second spacer to expand to plug the gap in the tricuspid valve depending on at least one of the plurality of pressures.

A further embodiment of the foregoing RVAD, wherein the controller is in communication with the first spacer and is configured to instruct the first spacer to expand to plug the gap in the pulmonary valve depending on the at least one of the plurality of pressures.

A further embodiment of the foregoing RVAD that includes a first sensor of the plurality of sensors for measuring a first pressure of the plurality of pressures with the first pressure being a pressure of blood within the pulmonary artery; a second sensor of the plurality of sensors for measuring a second pressure of the plurality of pressure with the second sensor being a pressure of blood within the right ventricle; the controller being in communication with the first and second sensors and configured to determine a first pressure gradient from the first pressure and second pressure; and a fifth alert that is triggered by the controller in response to an evaluation of the first pressure gradient signaling that the first valve is at least partially continuously open.

A further embodiment of the foregoing RVAD, wherein the tube is sterilized.

A right ventricular assist device for placement inside a heart and a pulmonary artery of a mammalian subject is disclosed herein that includes a tube having an inlet in a right side of the heart and an outlet in a pulmonary artery when the tube is in a proper position within the heart and the pulmonary artery, the tube extending from the pulmonary artery to at least a right atrium; a pump configured to pump blood from the inlet to the outlet; a first sensor positioned along the tube adjacent the outlet with the first sensor configured to be located within the pulmonary artery and measure a first pressure of blood within the pulmonary artery when the tube is in the proper position; a second sensor positioned along the tube and configured to be located within the right ventricle and measure a second pressure of blood within the right ventricle when the tube is in the proper position; a third sensor positioned along the tube and configured to be located within the right atrium and measure a third pressure of blood within the right atrium when the tube is in the proper position; a first spacer positioned along the tube between the first sensor and the second sensor with the first spacer being configured to deploy to plug a gap in a pulmonary valve of the heart; and a controller in communication with the first sensor, the second sensor, and the third sensor, the controller being configured to determine whether the first valve is at least partially continuously open to form the gap depending on the first pressure and the second pressure.

The RVAD of the preceding paragraph can optionally include, additionally and/or alternatively, any one or more of the following features, configurations and/or additional components:

A further embodiment of the foregoing RVAD, wherein the controller is in communication with the first spacer and is configured to instruct the first spacer to deploy to plug the gap in the first valve in response to the determination that the first valve is at least partially continuously open.

A further embodiment of the foregoing RVAD that includes a second spacer positioned along the tube between the second sensor and the third sensor with the second spacer being configured to deploy to plug a gap in a second valve of the heart.

A further embodiment of the foregoing RVAD, wherein the controller is configured to determine whether the second valve is at least partially continuously open to form the gap depending on the second pressure and the third pressure.

A further embodiment of the foregoing RVAD, wherein the controller is in communication with the second spacer and is configured to instruct the second spacer to deploy to plug the gap in the second valve in response to the determination that the second valve is at least partially continuously open.

A further embodiment of the foregoing RVAD, wherein the tube is sterilized.

A further embodiment of the foregoing RVAD, wherein the controller is in communication with the pump and is configured to instruct the pump to pump blood through the tube depending on at least one of the first pressure, the second pressure, and the third pressure.

A further embodiment of the foregoing RVAD that includes a first alert that is triggered by the controller in response to a comparison of the first pressure and the second pressure determining that the first pressure is within a first margin relative to the second pressure such that the tube is not in a proper position within the heart.

A further embodiment of the foregoing RVAD that includes a second alert that is triggered by the controller in response to a comparison of the second pressure and the third pressure determining that the second pressure is within a second margin relative to the third pressure such that the tube is not in the proper position within the heart.

A further embodiment of the foregoing RVAD that includes a display device in communication with the controller configured to visually display at least one of a measurement of the first pressure, a measurement of the second pressure, and a measurement of the third pressure.

A method of inserting a right ventricular assist device into a heart and a pulmonary artery of a mammalian subject is disclosed herein that includes inserting a distal end of a tube into the heart, the tube having an inlet along the tube, an outlet at the distal end of the tube, and a first sensor adjacent the distal end; measuring, by the first sensor, a first pressure of blood external to the tube at the first sensor; determining, from the first pressure, that the first sensor is within a right atrium of the heart; continuing to insert the tube into the heart, the tube having a second sensor proximal to the first sensor; measuring, by the first sensor, the first pressure of blood external to the tube at the first sensor and, by the second sensor, a second pressure of blood external to the tube at the second sensor; determining, from the first pressure and the second pressure, that the first sensor is within a right ventricle and the second sensor is within the right atrium; continuing to insert the tube into the heart and the pulmonary artery, the tube having a third sensor proximal to the second sensor on an opposite side from the first sensor; measuring, by the first sensor, the first pressure of blood external to the tube at the first sensor; by the second sensor, the second pressure of blood external the tube at the second sensor; and, by the third sensor, a third pressure of blood external to the tube at the third sensor; and from the first pressure, second pressure, and third pressure, determining that the tube is in a proper position such that the first sensor is within a pulmonary artery, the second sensor is within the right ventricle, and the third sensor is within the right atrium.

The method of the preceding paragraph can optionally include, additionally and/or alternatively, any one or more of the following features, configurations and/or additional components:

A further embodiment of the foregoing method that includes activating a pump to move blood through the tube from the inlet to the outlet positioned in the pulmonary artery.

A further embodiment of the foregoing method includes sterilizing the tube.

A further embodiment of the foregoing method includes continuously monitoring the first pressure, the second pressure, and the third pressure for changes that indicate that the first sensor is not in the pulmonary artery, the second sensor is not in the right ventricle, and the third sensor is not in the right atrium.

The above method(s) can be performed on a living animal or on a simulation, such as on a cadaver, cadaver heart, anthropomorphic ghost, simulator (e.g., with body parts, heart, tissue, etc. being simulated).

A method of monitoring an effectiveness of a right ventricular assist device within a heart and a pulmonary artery of a mammalian subject disclosed herein includes measuring a first pressure of blood by a first sensor positioned along a tube adjacent a distal end of the tube, the tube having an inlet and an outlet with the outlet being at the distal end; measuring a second pressure of blood by a second sensor positioned along the tube distant from the first sensor; measuring a third pressure of blood by a third sensor positioned along the tube distant from the second sensor on an opposite side from the first sensor; and determining, by a controller and from at least one of the first pressure, the second pressure, and the third pressure, whether the first sensor and the outlet are properly positioned in a pulmonary artery, the second sensor is properly positioned in the right ventricle, and the third sensor is properly positioned in the right atrium.

The method of the preceding paragraph can optionally include, additionally and/or alternatively, any one or more of the following features, configurations and/or additional components:

A further embodiment of the foregoing method that includes comparing the first pressure and the second pressure and triggering a first alert in response to the first pressure being within a first margin relative to the second pressure.

A further embodiment of the foregoing method, wherein triggering the first alert includes signaling that the tube is not properly positioned within the heart and the pulmonary artery.

A further embodiment of the foregoing method, wherein triggering the first alert further includes signaling that a first valve of the heart located between the pulmonary artery and the right ventricle is at least partially continuously open.

A further embodiment of the foregoing method comparing the second pressure and the third pressure and in response to the second pressure being within a second margin relative to the third pressure, triggering a second alert.

A further embodiment of the foregoing method, wherein triggering the second alert further includes signaling that the tube is not properly positioned within the heart and the pulmonary artery.

A further embodiment of the foregoing method, wherein triggering the second alert further includes signaling that a second valve of the heart located between the right ventricle and the right atrium is at least partially continuously open.

A further embodiment of the foregoing method that includes determining a first pressure waveform from the first pressure and, in response to the first pressure waveform being different from a pressure waveform of a properly functioning pulmonary artery, triggering a third alert.

A further embodiment of the foregoing method that includes determining a second pressure waveform from the second pressure and, in response to the second pressure waveform being different from a pressure waveform of a properly functioning right ventricle, triggering a fourth alert.

A further embodiment of the foregoing method that includes determining a third pressure waveform from the third pressure and, in response to the third pressure waveform being different from a pressure waveform of a properly functioning right atrium, triggering a fifth alert.

A further embodiment of the foregoing method that includes continuously measuring the first pressure and the second pressure and determining, dependent upon the first pressure and the second pressure, whether a first valve between the first sensor and the second sensor is at least partially continuously open.

A further embodiment of the foregoing method that includes activating a first spacer positioned along the tube between the first sensor and the second sensor, the first spacer configured to plug a gap in the first valve.

A further embodiment of the foregoing method, wherein activating the first spacer includes inflating a balloon to increase a cross-sectional area of the first spacer to plug the gap in the first valve.

A further embodiment of the foregoing method that includes continuously measuring the second pressure and the third pressure and determining, dependent upon the second pressure and the third pressure, whether a second valve between the second sensor and the third sensor is at least partially continuously open.

A further embodiment of the foregoing method that includes activating a second spacer positioned along the tube between the second sensor and the third sensor, the second spacer configured to plug a gap in the second valve.

A further embodiment of the foregoing method that includes pumping blood within the tube from the inlet to the outlet.

A further embodiment of the foregoing method that includes depending on at least one of the first pressure, the second pressure, and the third pressure, pumping blood at a variable flow rate from the inlet to the outlet.

A further embodiment of the foregoing method, wherein the blood is pumped at the variable flow rate to maintain the first pressure at a constant pressure.

A further embodiment of the foregoing method that includes reducing the flow rate of blood within the tube so that the first pressure does not exceed a threshold pressure.

A further embodiment of the foregoing method that includes pumping blood at a constant flow rate from the inlet to the outlet.

A further embodiment of the foregoing method that includes visually displaying at least one of the first pressure, the second pressure, and the third pressure on a display device.

A further embodiment of the foregoing method sterilizing the tube.

A further embodiment of the foregoing method, wherein the inlet is located within the right ventricle when the RVAD is properly positioned.

A further embodiment of the foregoing method, wherein the inlet is located within the right atrium when the RVAD is properly positioned.

The above method(s) can be performed on a living animal or on a simulation, such as on a cadaver, cadaver heart, anthropomorphic ghost, simulator (e.g., with body parts, heart, tissue, etc. being simulated).

A right ventricular assist device for placement at least partially inside a heart and a pulmonary artery of a mammalian subject is disclosed herein that includes a tube having an inlet configured to be positioned within the mammalian subject when the tube is in a proper position within the mammalian subject and an outlet configured to be located in the pulmonary artery when the tube is in the proper position; a pump connected to the inlet and configured to pump blood from the inlet to the outlet; a first sensor positioned along the tube adjacent the outlet, the first sensor configured to be located within the pulmonary artery when the tube is in the proper position within the heart and the pulmonary artery and measure a first pressure of blood exterior to the tube at a first measurement location of the first sensor; a second sensor positioned along the tube distant from the first sensor, the second sensor configured to be located within the right ventricle when the tube is in the proper position within the heart and the pulmonary artery and measure a second pressure of blood exterior to the tube at a second measurement location of the second sensor; a third sensor positioned along the tube distant from the first sensor and the second sensor, the third sensor configured to be located within the right atrium when the tube is in the proper position within the heart and the pulmonary artery and measure a third pressure of blood exterior to the tube at a third measurement location of the third sensor; and a controller in communication with the pump, the first sensor, the second sensor, and the third sensor, wherein the first sensor, the second sensor, and the third sensor convey measurements of the first pressure, the second pressure, and the third pressure, respectively, to the controller and the controller is configured to instruct the pump to pump blood through the tube depending on at least one of the measurements of the first pressure, the second pressure, and the third pressure.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A right ventricular assist device for placement at least partially inside a heart and a pulmonary artery of a mammalian subject, the right ventricular assist device comprising:
a tube having an inlet configured to be positioned within the mammalian subject when the tube is in a proper position within the mammalian subject and an outlet configured to be located in the pulmonary artery when the tube is in the proper position;
a pump connected to the inlet and configured to pump blood from the inlet to the outlet;
a plurality of sensors having measurement locations along the tube to measure a plurality of pressures of blood exterior to the tube at the measurement locations, wherein the measurement locations of the plurality of sensors are spaced apart from one another along the tube such that, when the tube is in the proper position within the heart and the pulmonary artery, at least one measurement location is within each of a right atrium, a right ventricle, and the pulmonary artery; and
a controller in communication with the pump and the plurality of sensors, wherein the plurality of sensors convey the plurality of pressures to the controller, and wherein the controller is configured to instruct the pump to pump blood through the tube depending on at least one of the plurality of pressures.

2. The right ventricular assist device of claim 1, wherein the controller is configured to instruct the pump to vary a flow rate of blood pumped through the tube.

3. The right ventricular assist device of claim 2, wherein the controller is configured to instruct the pump to vary the flow rate of blood to maintain a first pressure representing a pressure of blood in the pulmonary artery above a first threshold pressure, and wherein the controller is configured to instruct the pump to vary the flow rate of blood to maintain a first pressure representing a pressure of blood in the pulmonary artery below a second threshold pressure.

4. The right ventricular assist device of claim 1, wherein the inlet is configured to be positioned within a right ventricle or a right atrium of the heart.

5. The right ventricular assist device of claim 1, wherein the plurality of pressure sensors comprises:
a first sensor positioned along the tube adjacent the outlet, the first sensor configured to be located within the pulmonary artery when the tube is in the proper position within the heart and the pulmonary artery and measure a first pressure of the plurality of pressures;
a second sensor positioned along the tube distant from the first sensor, the second sensor configured to be located within the right ventricle when the tube is in the proper position within the heart and the pulmonary artery and measure a second pressure of the plurality of pressures; and
a third sensor positioned along the tube distant from the first sensor and the second sensor, the third sensor configured to be located within the right atrium when the tube is in the proper position within the heart and the pulmonary artery and measure a third pressure of the plurality of pressures.

6. The right ventricular assist device of claim 1, wherein the plurality of sensors comprises:
a first port at a first measurement location within the pulmonary artery when the tube is in the proper position within the heart and the pulmonary artery;
a first sensor in fluid communication with the first port and configured to measure a first pressure of blood exterior to the tube at the first port;
a first lumen extending at least partially within the tube from the first port to the first sensor;
a second port at a second measurement location within the right ventricle when the tube is in the proper position within the heart;
a second sensor in fluid communication with the second port and configured to measure a second pressure of blood exterior to the tube at the second port;
a second lumen extending at least partially within the tube from the second port to the second sensor;
a third port at a third measurement location within the right atrium when the tube is in the proper position within the heart;
a third sensor in fluid communication with the third port and configured to measure a third pressure of blood exterior to the tube at the third port; and
a third lumen extending at least partially within the tube from the third port to the third sensor;
wherein the first sensor, the second sensor, and the third sensor are incorporated into one pressure measurement device in communication with the controller, the one pressure measurement device configured to measure at least one of the first pressure, the second pressure, and the third pressure.

7. The right ventricular assist device of claim 6, wherein the first pressure, second pressure, and third pressure are conveyed to the controller, and the right ventricular assist device further comprising:
a first alert that is triggered by the controller in response to a comparison of the first pressure to the second pressure revealing that the first pressure is within a first margin as compared to the second pressure; and
a second alert that is triggered by the controller in response to a comparison of the second pressure to the third pressure revealing that the second pressure is within a second margin as compared to the third pressure;
wherein the first alert is signaling one of the following: the tube is not in the proper position within the heart and the pulmonary artery, and a first valve between the pulmonary artery and the right ventricle is at least partially continuously open; and
wherein the second alert is signaling one of the following: the tube is not in the proper position within the heart and the pulmonary artery, and a second valve between the right ventricle and the right atrium is at least partially continuously open.

8. The right ventricular assist device of claim 6, wherein the first pressure, second pressure, and third pressure are conveyed to the controller and the controller is configured to determine at least one of a first pressure waveform from the first pressure, a second pressure waveform from the second pressure, and a third pressure waveform from the third pressure.

9. The right ventricular assist device of claim 8, further comprising:
a third alert that is triggered by the controller in response to a comparison of the first pressure waveform to the second pressure waveform revealing that the first pressure waveform is within a first waveform margin as compared to the second pressure waveform; and
a fourth alert that is triggered by the controller in response to a comparison of the second pressure waveform to the third pressure waveform revealing that the second pressure waveform is within a second waveform margin as compared to the third pressure waveform.

10. The right ventricular assist device of claim 8, wherein:
the controller, depending on the first pressure waveform and the second pressure waveform, determines if a first valve is open between the pulmonary artery and the right ventricle; and
the controller, depending on the second pressure waveform and the third pressure waveform, determines if a second valve is open between the right ventricle and the right atrium.

11. The right ventricular assist device of claim 1, further comprising:
a first spacer positioned along the tube and configured to be between the pulmonary artery and the right ventricle when the tube is in the proper position, the first spacer configured to plug a gap in the pulmonary valve; and
a second spacer positioned along the tube and configured to be between the right ventricle and the right atrium when the tube is in the proper position, the second spacer configured to plug a gap in the tricuspid valve.

12. The right ventricular assist device of claim 11, wherein the first spacer is one of an inflatable balloon, a covered braid, a covered wireform, and a temporary valve, and wherein the second spacer is one of an inflatable balloon, a covered braid, a covered wireform, and a temporary valve.

13. The right ventricular assist device of claim 11, wherein the controller is in communication with the first spacer and is configured to instruct the first spacer to expand to plug the gap in the pulmonary valve depending on the at least one of the plurality of pressures, and wherein the controller is in communication with the second spacer and is configured to instruct the second spacer to expand to plug the gap in the tricuspid valve depending on at least one of the plurality of pressures.

14. A right ventricular assist device for placement at least partially inside a heart and a pulmonary artery of a mammalian subject, the right ventricular assist device comprising:
a tube having an inlet configured to be positioned within the mammalian subject when the tube is in a proper position within the mammalian subject and an outlet configured to be located in the pulmonary artery when the tube is in the proper position;
a pump connected to the inlet and configured to pump blood from the inlet to the outlet;
a first sensor positioned along the tube adjacent the outlet, the first sensor configured to be located within the pulmonary artery when the tube is in the proper position within the heart and the pulmonary artery and measure a first pressure of blood exterior to the tube at a first measurement location of the first sensor;
a second sensor positioned along the tube distant from the first sensor, the second sensor configured to be located within the right ventricle when the tube is in the proper position within the heart and the pulmonary artery and measure a second pressure of blood exterior to the tube at a second measurement location of the second sensor;
a third sensor positioned along the tube distant from the first sensor and the second sensor, the third sensor configured to be located within the right atrium when the tube is in the proper position within the heart and the pulmonary artery and measure a third pressure of blood exterior to the tube at a third measurement location of the third sensor; and
a controller in communication with the pump, the first sensor, the second sensor, and the third sensor, wherein the first sensor, the second sensor, and the third sensor convey measurements of the first pressure, the second pressure, and the third pressure, respectively, to the controller and the controller is configured to instruct the pump to pump blood through the tube depending on at least one of the measurements of the first pressure, the second pressure, and the third pressure.

15. The right ventricular assist device of claim 14, further comprising:
a first port at the first measurement location within the pulmonary artery when the tube is in the proper position within the heart and the pulmonary artery;
a first lumen extending at least partially within the tube from the first port to the first sensor;
a second port at the second measurement location within the right ventricle when the tube is in the proper position within the heart;
a second lumen extending at least partially within the tube from the second port to the second sensor;
a third port at the third measurement location within the right atrium when the tube is in the proper position within the heart; and
a third lumen extending at least partially within the tube from the third port to the third sensor.

16. The right ventricular assist device of claim 14, wherein the controller is configured to instruct the pump to vary the flow rate of blood to maintain the first pressure representing the pressure of blood in the pulmonary artery above a first threshold pressure and, wherein the controller is configured to instruct the pump to vary the flow rate of blood to maintain a first pressure representing the pressure of blood in the pulmonary artery below a second threshold pressure.

17. A right ventricular assist device for placement at least partially inside a heart and a pulmonary artery of a mammalian subject, the right ventricular assist device comprising:
a tube having an inlet configured to be positioned within the mammalian subject when the tube is in a proper position within the mammalian subject and an outlet configured to be located in the pulmonary artery when the tube is in the proper position;
a pump connected to the inlet and configured to pump blood from the inlet to the outlet;
a plurality of sensors having measurement locations along the tube to measure a plurality of pressures of blood exterior to the tube at the measurement locations, wherein the measurement locations of the plurality of sensors are spaced apart from one another along the tube such that, when the tube is in the proper position within the heart and the pulmonary artery, at least one measurement location is within each of a right atrium, a right ventricle, and the pulmonary artery;
a controller in communication with the pump and the plurality of sensors, wherein the plurality of sensors convey the plurality of pressures to the controller, and wherein the controller is configured to instruct the pump to pump blood through the tube depending on at least one of the plurality of pressures;
a first spacer positioned along the tube and configured to be between the pulmonary artery and the right ventricle when the tube is in the proper position, the first spacer configured to plug a gap in the pulmonary valve; and
a second spacer positioned along the tube and configured to be between the right ventricle and the right atrium when the tube is in the proper position, the second spacer configured to plug a gap in the tricuspid valve.

18. The right ventricular assist device of claim 17, wherein the first spacer is one of an inflatable balloon, a covered braid, a covered wireform, and a temporary valve, and wherein the second spacer is one of an inflatable balloon, a covered braid, a covered wireform, and a temporary valve.

19. The right ventricular assist device of claim 17, wherein the controller is in communication with the first spacer and is configured to instruct the first spacer to expand to plug the gap in the pulmonary valve depending on the at least one of the plurality of pressures, and wherein the controller is in communication with the second spacer and is configured to instruct the second spacer to expand to plug the gap in the tricuspid valve depending on at least one of the plurality of pressures.

20. The right ventricular assist device of claim 17, wherein the controller is configured to instruct the pump to vary a flow rate of blood pumped through the tube depending on at least one of the plurality of pressures.
